(19) United States
(12) Patent Application Publication
(10) Pub. No.: US 2005/0180961 A1
(43) Pub. Date: Aug. 18, 2005

(54) Probiotics and oral tolerance

(76) Inventors: Sophie Pecquet, Lausanne (FR); Guenolee Prioult, Sainte-Foy (CA)

Correspondence Address: Robert M Barrett;Bell Boyd & Lloyd, Attorneys for Applicants, P O Box 1135, Chicago, IL, 60690-1135, US

(21) Appl. No.: 10/515,758

(22) PCT Filed: May 22, 2003
(86) PCT No.: PCT/EP03/05354

(30) Foreign Application Priority Data
May 24, 2002 (EP) 02011444.3

(51) Int. Cl. A23K 1/165; A61K 47/00

(52) U.S. Cl. 424/93.45; 424/439; 424/442

(57) ABSTRACT

The invention refers to food products comprising at least a mean to promote the oral tolerance phenomenon (Lactobacillus paracasei), to food products comprising at least a mean to maintain the oral tolerance phenomenon (Bifidobacterium lactis), and to food products comprising means to promote and maintain the oral tolerance phenomenon (combination of Lactobacillus and Bifidocateria). The food product is intended for infants, babies, children, and also for pets. The invention refers to food products which are not intended for allergic populations.

### BACKGROUND OF THE INVENTION

Cow's milk allergy or milk hypersensitivity is very common in infants and usually disappears by the age of two or three years, but may occasionally be lifelong. It is the most common disease in infants, with an incidence of 0.5 to 3% in full term infants and 3 to 5% in preterm infants. This allergy can cause rash, hives, redness around the mouth, runny nose, sneezing, colic, diarrhoea, vomiting, anaphylaxis, or more generally digestive troubles. It could also be associated in some cases of infant sudden death.

Milk hypersensitivity should be differentiated from lactose intolerance, which is an intolerance to milk as a result of congenital deficiency of the lactase enzyme.

Cow's milk allergy is caused, in most cases, by the α-lactoglobulin and the β-lactoglobulin allergens. It can also be caused by casein and/or albumin, which are potentially allergenic lactic proteins also present in cow's milk. The allergy, when developed, is caused by an hypersensitivity reaction of the immune system to the above-mentioned proteins. In the first step of the process (sensitisation), the immune system recognises the protein as an antigen when it enters the body, and this generates an immune response consisting of specific antibodies or specifically sensitised T lymphocytes. In the second step, if the antibody is an IgE antibody, it will respond to the presence of the allergen by generating an inflammatory reaction, which is the allergy.

The mechanism of this type of allergy can be explained as follows: the IgE antibodies appear on the surface of cells, including circulating basophils. When the interaction allergen/IgE occurs, the cells presenting the IgE/allergen couple generates and releases chemical mediators, including histamine. This phenomenon leads in pathologic effects, such as vasodilatation, locally or systemically.

Usually, this milk hypersensitivity appears with the first attempt of food diversification when the infant is first presented to the cow's milk.

WO 01/97822 to Oy aboatech AB discloses the use of a probiotic bacteria, for example Lactobacillus casei ssp. rhamnosus, for a preparation of a composition useful for primary prevention of atopic diseases in an infant at high risk of atopic diseases. It is an attempt to prevent allergies in a specific population which is the high risks of allergy population.

WO 01189541 to Compagnie Gervais Danone discloses the use of a Lactobacillus casei in a composition for oral administration to enhance immunity specific to pathogenic micro-organisms. It is particularly intended for pathogens affected respiratory system, and the composition can be a food or a food supplement.

However, a need exists for a mean of preventing atopic diseases such as cows milk allergies and/or maintaining the oral tolerance acquired by the infant, either by himself or with the help of said aforementioned mean.

### SUMMARY OF THE INVENTION

Accordingly, the present invention provides the use of a Bifidobacterium strain for the manufacture of a food product for the maintenance of oral tolerance to antigens. The invention further provides the use of a Lactobacillus and a Bifidobacteria for the manufacture of a food product for the promotion and maintenance of oral tolerance to antigens.

The invention refers to food products which are not intended for allergic populations.

### FIGURES

FIG. 1 shows the decrease of BLG-specific antibody titers (IgE, IgG1 and Ig2a) in serum of mice (conventional (CV), monoassociated and germfree (GF)) killed 28 days (A) or 50 days (B) after oral feeding with whey proteins

FIG. 2 shows the production of IFN-γ, IL-10, IL-5 and IL-4 of spleen cells in vitro. Mice (Conventional (Conv), monoassociated and germfree (GF)) were orally fed whey proteins (black bars) or saline water (open bars) before being subcutaneously injected with 100 μg of BLG and 100 μg of OVA in Al(OH)₃ and sacrificed 28 days (A) or 50 days (B) after gavage. Mice sacrificed at day 50 received two additional injections at day 21 and 35.

### DETAILED DESCRIPTION OF THE INVENTION

In the context of the present invention, the term “food product” is intended to encompass any consumable matter. Hence, it may be a product intend for the consumption by humans, but the term also encompasses products to be consumed by animals for example pets, such as dogs, cats, rabbits, guinea pigs, mice, rats, birds (for example parrots), reptiles and fish (for example goldfish). However, the term also includes food to be consumed other domesticated animals, such as livestock for example, cattle, horses, pigs, sheep, goats, buffaloes, camels, and the like. This term is also intended to include any infant formula, baby formula, infant and baby follow-up formula, and the like.

It has to be understood that when talking about cow's milk allergy or cow's milk hypersensitivity, we mean any food allergy or hypersensitivity, and more generally any atopic disease. Our argumentation is mostly developped around this allergy because cow's milk is in most cases the first food product encountered by infants; however, we do not limit the scope of the present patent application to this disease.

Suprisingly, we have found that the addition of probiotics to food products could be is used as an adjuvant to promote oral tolerance, and to maintain it. Specifically, we have found that lactic acid bacteria strains belonging to the Lactobacillus genus, and in particular Lactobacillus paracasei strains are able to promote the induction of oral tolerance, and is particularly suitable to promote the oral tolerance to cow's milk proteins.

In a preferred embodiment of the invention, the Lactobacillus used is Lactobacillus paracasei CNCM I-2116. These microorganisms have been shown to exhibit inter alia the following properties: they are gram positive, catalase negative, NH₃ from arginine negative, and CO₂ production negative. They produce L(+) lactic acid and are capable to grow in the presence of bile salts in a concentration of up to 0.4% o.

According to the invention, the lactic acid bacteria used can be added as an adjuvant or a supplement in particular to infant formulas used from birth to the introduction of antigens (such as cow's milk antigens) to the alimentation, in order to promote the oral tolerance to the said antigens. As the lactic acid bacteria will not be a dominant flora of the infant's intestine, large amounts of the microorganism should be incorporated to the formulae, for example 10⁵ to 10⁸ cfu/mL of reconstituted formulae or cfu/g of food product, more preferably 10⁵ to 10⁷, and in a preferred embodiment 10⁶ cfu/mL of reconsituted formula. Thus, the lactic acid bacteria will be ingested several times a day, leading to a quantity of this micro-organism in the gut microflora constantly sufficient to be effective for the purpose of the present invention.

The aforementioned lactic acid bacteria, belonging to the Lactobacillus genus, being in particular a Lactobacillus paracasei and in a preferred embodiment Lactobacillus paracasei CNCM I-2116 can also be incorporated in pet food products in order to promote oral tolerance. It can be incorporated in chunk products or croquettes preferably, but it can also be incorporated in humid products, such as cans, for example. It can also be added to liquid formulas for pets, such as milk for kittens or puppies, among others, and can as well be added to chew products for pets.

The supplementation of pet food by this micro-organism can be comprised, for example, between 10⁴ and 10⁸ cfu/g for non humid products, 10⁵ to 10⁸ cfu/g for humid products, and 10⁵ to 10⁸ cfu/g for liquid products, these amounts not being limitative.

We further investigated the proteolytic activity of Lactobacillus paracasei CNCM I-2116 and its potential role in the degradation of the most important allergen in milk: β-lactoglobulin.

Proteolytic system of lactic acid bacteria can be divided into three groups on the basis of their function:
i) proteinases that breakdown whole protein to peptides,
ii) peptidases that degrade peptides and
iii) transport systems that translocate the breakdown products across the cytoplasmic membrane.

Proteases are present outside the bacterial cells, whereas most of peptidases are found in the cytoplasm. In the study, peptidases from cytoplasmic extract have been used to degrade native beta-lactoglobulin (BLG) and its tryptic-chymotriptic (TC) peptides. L. paracasei-associated enzymes hydrolyze mainly acidic TC peptides while basic peptides are slightly degraded and BLG not at all. Since, BLG TC hydrolysate was shown to be composed mainly of acidic peptides (62%), their degradation with L. paracasei enzymes is of great interest. However, the proteolytic activity is certainly limited to amino- or di-peptidases because L. paracasei degradation releases peptides with molecular weights mainly lower 500 Da, as observed by size exclusion chromatography.

The basic fraction is also degraded by amino- and di-peptidase activities and we have also observed that peptides with molecular weights higher than 2000 Da appear in the fraction after L. paracasei degradation. Although not wishing to be bound by theory, strong hydrophobic interactions between peptides could explain this observation because basic TC peptides have been shown to be highly hydrophobic by C18 chromatography.

The degradation of acidic TC peptides with L. paracasei extract lead to peptides with immunosuppressive properties, these effects being essential for oral tolerance induction. Well-characterised mechanisms for the induction of tolerance include clonal deletion, clonal anergy and active suppression via the induction of regulatory T cells. The type I-regulatory T cells (Tr-1) have a low proliferation capacity and suppress naïve and memory T helper type 1 and 2 responses due to their ability to produce high levels of immunosuppressive cytokines like IL-10. IL-10 was found to downregulate CD54 expression, CD80 and CD86 which function as important costimulatory molecules for T cell activation. Interestingly IL-10 was found to be up-regulated in response to L. paracasei-degraded acidic peptides indicating an effect of these peptides to induce oral tolerance to BLG by a mechanism of active suppression.

The potential beneficial effect of L. paracasei-degraded acidic peptides on oral tolerance phenomenon, underlined in BLG-naïve mice, is reinforced by the results obtained in BLG-primed mice (BLG-tolerant mice). A suppression of lymphocyte proliferation has also been observed with splenocytes from BLG-primed mice indicating a real capability of degraded peptides to induce, maintain and reinforce hyporesponsiveness of T cells.

L. paracasei CNCM I-2116 stimulates regulatory T cells through its proteolytic activity and liberation of bioactive peptides. We have also demonstrated an immunosuppressive effect of the cytoplasmic content of L. paracasei. These findings strongly suggest that bacteria such as L. paracasei CNCM I-2116 induce an immunosuppressive activity, either directly via cell to cell interactions or via degradation of antigens and liberation of cytoplasmic content after death.

We have also discovered that strains of the Bifidobacterium genus have a specific effect on the oral tolerance maintenance. Consequently, it is a purpose of the invention to add Bifidodacteria to foodstuffs as a supplement or an adjuvant, especially to populations wherein oral tolerance has already bee induced. In particular, such population can be a breast-fed infant population; indeed, part of the allergens eaten by the mother are given to the child via the mother's milk; in this population, oral tolerance has been promoted via breast-feeding, and this tolerance can be maintained by giving to the infant an effective amount of Bifidobacteria having the aforementionned property.

It is well known that some ants, who do not develop cows milk hypersensitivity when they first encounter cow's milk nevertheless develop some food hypersensitivity when they encounter other food ingredients, for example at the age of 8 months. Although not wishing to be bound by theory, we believe that they have induced an oral tolerance which has not been maintained or not been maintained properly. Consequently, a purpose of the invention is to maintain an oral tolerance previously Consequently, a purpose of the invention is to maintain an oral tolerance previously induced, by means of adding to the alimentation a Bifidobacteria having the ability to do so.

It might be useful to give the bifidus to the child or the infant at the beginning of the process.

In a preferred embodiment of the invention, the Bifidobacteria is a Bifidobacterium lactis, and can be in the best mode of realisation the well-known Bb12 strain, ATCC 27536 which can be obtained from Hansen (Chr. Hansen A/S, 10-12 Boege Alle, P.O. Box 407, DK-2970 Hoersholm, Danemark). As the nomenclature has changed several time, Bb12 can also be found in literature and in commercialised products as B. bifidum and B. animalis.

This microorganisms have been shown to exhibit inter alia the following properties: they are gram +, non mobitile and nonsporing rods, resistant to gastrointestinal acidity and bile salts. They are catalase negtive, and produce only L(+) lactic acid and not its D(−) isomer. They can utilize the following carbohydrates: ribose, saccharose, D-glucose, D-raffinose, maltose, melibiose, amygdalin and beta-gentobiose.

They can be added to any kind of infant, baby, or childhood food product, such as malted milk, infant formulae, follow up formulae, baby cereals and the like. Sometimes, diversification of the infant alimentation can occur as early of four months of life, and the bifidobacteria can be incorporated to the infant food given to children aged four months and older. As milk hypersensitivity in the majority disappears at the age of 2 to 3 years, supplementation of infant and baby food with Bifidobacterium to maintain oral tolerance can be done in every food product for infants and babies from 4 months to 3 years. But supplementation can also be realised in food products not specifically aimed for infant or baby nutrition and nevertheless being part of their alimentation, for example milk, yoghurt, curd, cheese, fermented milks, milk based fermented products, ice-creams, fermented cereal based products, or milk based products, among others.

The amount of microorganism added to the infant food product, baby food product or food product in general should be of from 10⁵ to 10⁸ cfu/mL of reconsituted formula or cfu/g of food product, preferably 10⁵ to 10⁷, and in a most preferred embodiment 10⁶. However these amounts should not be considered as limitative and should be adapted to the aimed population, for example based on the weight and age of the infant or baby, or specific populations such as population having specific diseases, for example infants, babies or children having gut diseases, acute diarrheas or inflammatory syndroms, among others.

The aforementioned Bifidobacteria, being in particular a Bifidobacterium lactis and in a preferred embodiment Bb12 Bifidobacterium lactis ATCC 27536 can also be incorporated in pet food products in order to maintain oral tolerance. It can be incorporated in chunk products or croquettes preferably, but it can also be incorporated in humid products, such as cans, for example. It can also be added to liquid formulas for pets, such as milk for kittens or puppies, among others, and can as well be added to chew products for pets.

The supplementation of pet food by this micro-organism can be comprised, for example, between 10⁴ and 10⁸ cfu/g for non humid products, 10⁵ to 10⁸ cfu/g for humid products, and 10⁵ to 10⁸ cfu/g for liquid products, these amounts not being limitative.

We have shown that a cell-free extract of B. lactis Bb12 hydrolyzes tryptic-chymotryptic peptides of BLG and thereby both decreases their IgE recognition and increases their in vitro stimulation of Th1 cells. Acidic TC peptides are better splenocyte proliferation inducers than basic peptides. However, when further hydrolyzed by B. lactis cell-free extract, acidic and basic peptides exhibit similar stimulating effects. This stimulating effect is more pronounced in naïve mice than in BLG tolerant mice.

The relative resistance of proteins such as BLG to proteases allows portions of them to remain intact after digestion, making them potentially highly allergenic. It is generally accepted that hydrolysis of proteins by trypsin and/or chymotrypsin decreases their allergenicity. Consistent with this finding, we observed that the IgE binding capacity of tryptic-chymotryptic peptides of BLG is lower than for native BLG.

Although not wishing to be bound by theary, we believe that the mechanism of intervention by these potential anti-allergy strains appears to be regulation of the pro-Th 1/anti-Th2 response through the enhancement of IFN-γ production, a Th1 cell-related cytokine. IFN-γ is a potent immunoregulatory and inflammatory cytokine which has been found at low levels in neonates and deficient in patients with food allergy. IFN-γ plays a crucial role in food allergy prevention by inhibiting Th2 differentiation decreasing levels of IL-4 and hence inhibition of IgE production. We observed that cell free extract alone from B. lactis Bb12 induces IFN-γ production by splenocytes. These findings strongly suggest that bacteria such as B. lactis Bb12 enhance the Th1 profile either directly or via degradation of potentially allergenic proteins after cell lysis and liberation of cytoplasmic contents into the in tract.

In addition to the previously mentioned effects of IFN-γ, it has been reported that IFN-γ may increase absorption of intact antigens across the gut mucosa, suggesting a potential unfavorable effect of B. lactis Bb12 for subjects with hyperresponsiveness to milk proteins. However, antigen transport across Peyer's patches has also been enhanced by IFN-γ. Since Peyer's patches are the site of secretory IgA formation following oral antigen exposure, local stimulation of IFN-γ production by B. lactis Bb12 may inhibit penetration of potentially harms dietary antigens via the mechanism of immune exclusion. In agreement with this finding, teased fecal IgA levels have been observed in infants fed B. lactis Bb12-containing follow-up formula.

Our results show that B. lactis Bb12 exhibits anti-allergy properties through a mechanism of immune deviation enhancing a pro-Th1/anti-Th2 cell response and through mechanisms of hydrolysis and immune exclusion of potentially harmful dietary antigens. We investigated whether Bb12 can induce IFN-γ production in young mice never previously exposed to BLG (‘naïve ’ mice). Production of IFN-γ in response to B. lactis-degraded fractions is more apparent with splenocytes of naïve mice than of mice rendered tolerant by feeding a BLG-enriched diet, suggesting that B. lactis Bb12 maintains the Th1 response in bottle-fed neonates and in older babies counterbalancing the Th2-polarized immunity of newborns.

Preferably, a combination of a Lactobacillus and a Bifidobacteria to both promote and maintain oral tolerance, in particular to cow's milk will be use. In a preferred embodiment, the food product could comprise Lactobacillus paracasei and Bifidobacterium lactis to achieve the desired purpose. For example, the combination could comprise Lactobacillus paracasei CNCM I-2116 and Bifidobacterium lactis ATCC 27536. The means to promote and maintain oral tolerance can be a combination of one or several Lactobacilli with one or several Bifidobacteria. For example, it can be a combination of Lactobacillus paracasei, Lactobacillus thawed and Bifidobacterium lactis. Depending on which population will ingerate the food supplemented with the microorganisms, the proportion of Lactobacilli compared to Bifidobacteria might vary. For example, for a population of infants which have not yet been in contact with cow's milk, the Lactobacilli:Bifidobacteria ratio could be comprised between 10:1 and 1:1. For a population of infants, babies or children having previously ingested cow's milk (or any other allergen or antogen), the Lactobacilli:Bifidobacteria ratio could be comprised between 1:10 and 1:1.

If more than one Bifidobacterium is incorporated to the food product, the ratio of one Bifidobacteria compared to the other can be from 0.01 to 99.9%, and the same applies if there is more than one Lactobacillus in the preparation.

The food product in which the combination of micro-organisms according to the invention is added can be any food product listed in the description of the first aspect of the invention and the second aspect of the invention, as well as any other food product, for example chocolate, chocolate powder, spreads, pastries, jellies, jams, biscuits, snacks, juices, dairy products, breakfast cereals, and more generally any food product eaten by infants, babies or children, as well as any food product eaten by pets.

The amount of microorganisms in the foodstuff according to the invention is preferably comprised between 10⁴ and 10⁸ cfu/g of food product or cfu/mL of reconstituted product when incorporated to humid or liquid products, and 10⁴ to 10⁹ cfu/g of food product when incorporated to non-humid or less humid product. The ratio Lactobacilli:Bifidobacteria does not interfere on the quantity of microorganisms added into the food product.

Particularly, microorganisms of the invention, alone or in combination, help to prevent and maintain tolerance of ingested antigens. It is particularly suitable for food allergies, such as allergies to shellfish comprising shrimp, crayfish, lobster and crab, to peanuts, eggs, tree nuts (for example walnuts or cashew), soy, wheat, fish, and any other known food allergy. It is also suitable for other allergies, such as allergies to acarids, pollens or dusts, among others.

Probiotics may also be used in the manufacture of a food product to prevent from the risk of rejecting transplants. Such food product can comprise at lea one member of the lactic acid bacteria family to induce oral tolerance, and/or at least one member of the Bifidobacteria genus to maintain an oral tolerance previously induced. The lactic acid bacteria stains belonging to the Lactobacillus genus are preferred, in particular Lactobacillus paracasei, and more particularly Lactobacillus paracasei CNCM I-2116. The Bifidobacteria used is preferably a Bifidobacterium lactis, and more particularly the Bb 12 ai ATCC 27536.

Indeed, although not wishing to be bound by theory, we believe that these two bacteria have the ability to specifically stimulate the immune system by promoting oral tolerance. Consequently, the mechanism developed by the immune system together with these bacteria, alone or in combination, moving away allergies is not specific to cow's milk allergy or hypersensitivity, and this mechanism can help patients in need of a transplantation or having been transplanted not to reject their transplant. An aspect of the present invention is so to prevent the risk of graft versus host diseases appearance by helping to the induction and the maintain of the oral tolerance to the graft, prior the transplantation and thereafter, in order to help patients to support transplantation, from an immunological point of view, by acquiring oral tolerance to the antigen that will be transplanted.

The microorganisms added to the food products according to the invention are preferably alive but can also be dead or can be inactivated for example by lyophilisation. Indeed, the en membrane of the dead bacteria would seem to activate the immune system, at least partly, in the same way than the alive bacteria. Consequently, in any embodiment of the invention, the bacteria or combination of bacteria can be achieved with dead organisms. In this case, the amount of microorganisms incorporated to the food product is preferably enhanced compared to the amounts given above, from 5% to 250%.

### EXAMPLES

The following examples are illustrative of some of the products and methods of mug the same falling within the scope of the present invention. They are not to be considered in any way limitative of the invention. Changes and modifications can be made with respect to the invention. That is, the skilled person will recognise many variations in these examples to cover a wide range of formulas, ingredients, processing, and mixtures to rationally adjust the naturally occurring levels of the compounds of the invention for a variety of applications.

### Example 1

### Induction of Oral Tolerance

3 groups of 20 germ-free female mice C3H/HeJ 3 to 5 weeks old and one group of conventional female mice BALB/c 3 weeks old were used in this exponent. Two probiotic strains were used in the study: one isolated from faces of healthy babies was from the Nestlé Culture Collection (Lausanne, Switzerland): Lactobacillus paracasei from the Nestlé Culture Collection (Lausanne, Switzerland): Lactobacillus paracasei NCC 2461 (CNCM 1-2116) and one was purchased from Chris Hansen (France) and is of human origin: Bifidobacterium lactis Bb12 NCC 362.

The germ free mice were inoculated by the oral route with an intragastric tube with 0.3 mL of a 24 hours bacterial culture containing around 5.10⁸ cfu/mL of one out of the L. paracasei or B. lactis strains. Oral induction was induced two weeks after bacterial feeding. In each group, 10 mice were given oral administration of 3 mg/g body weight whey proteins to induce oral tolerance. Whey proteins were obtained by ultrafiltration of acid whey, and the protein content was 80%, approximately 62% of which was β-lactoglobulin. The remaining 10 mice orally received a single feeding of saline water as negative control (non-tolerized mice).

Five days later, all mice were immunised subcutaneously with 100 μm β-lactoglobulin (BLG) 3 times crystallised and 100 μg ovalbumin grade V (OVA). Mice were sacrificed 28 or 50 days after gavage to assess induction and maintenance of tolerance, respectively. At each sacrifice, 5 whey-protein fed and 5 water-fed mice were killed. Mice sacrificed at 50 days received two additional subcutaneous injections of BLG and OVA at day 21 and 35.

The amount of BLG-specific IgE and BLG-specific IgG1 and IgG2a levels in serum were determined by ELISA.

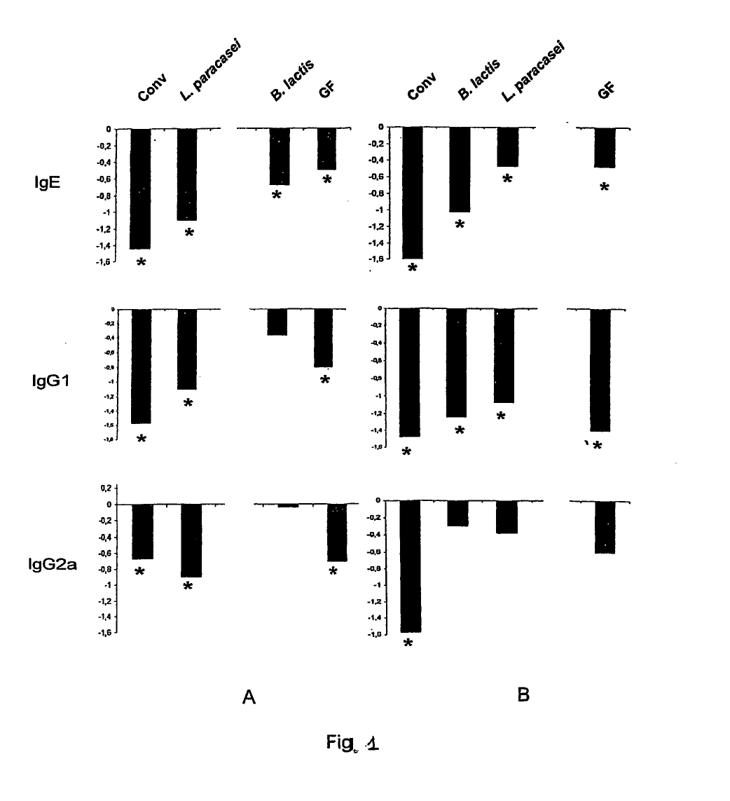

The induction of oral tolerance in conventional, germfree and monoassiociated mice were assessed in mice sacrificed 28 days after oral administration of whey proteins and immunised with BLG: see FIG. 1A. The results clearly showed that whey proteins feeding significantly suppressed (P<0.05) the BLG-specific IgE, IgG1 and IgG2a antiboby responses in conventional, germfree and L. paracasei-associated mice indicating a Th1- and Th2-type humoral responses suppression. In mice associated with B. lactis, only anti-BLG IgE response was significantly suppressed: anti-BLG IgG1 response was suppressed but not significantly (P<0.2). Hence, only the Th2-type humoral responses were sensitive to tolerance in this group of mice.

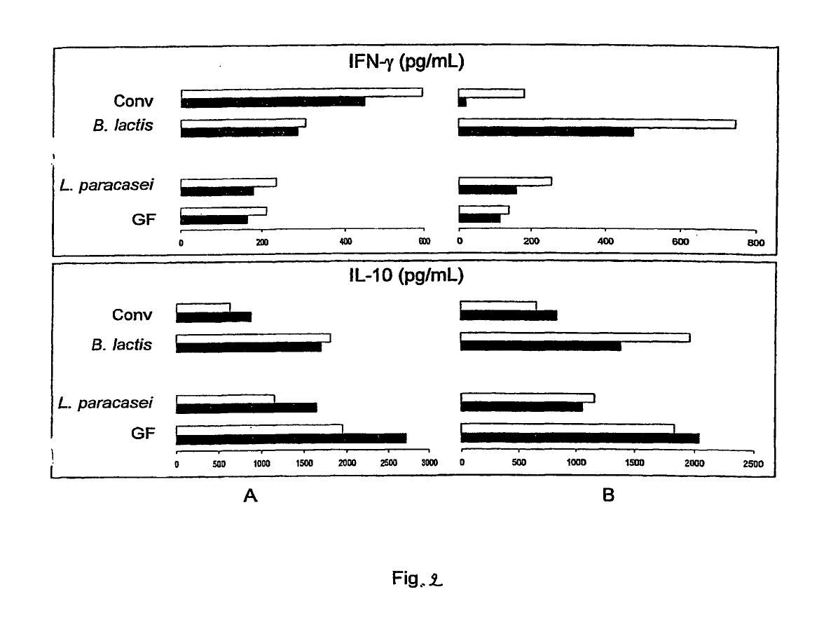

Induction of tolerance in terms of spleen cells proliferation and cytokine production: to further confirm that tolerance induction is modulated by probiotics, spleen cells were cultured in vitro and both cell proliferation (table 1) and cytokine profiles in culture supernatants were examined (FIGS. 2A)

TABLE 1
Proliferative response of BLG- or PHA-activated splenocytes from conventional
(Conv), monoassociated and germfree (GF) mice killed 28 days or 50 days after oral
feeding with whey proteins.

| | Proliferative index | | | | | | | | | |
|---|---|---|---|---|---|---|---|---|---|---|
| | Proliferation (cpm) | | | | | | Stimulation index | | | |
| | Day 28 | | | Day 50 | | | Day 28 | | Day 50 | |
| | Ag(−) | BLG | PHA | Ag(−) | BLG | PHA | BLG | PHA | BLG | PHA |
| Conv | 212 | 3.174 | 70.541 | 311 | 7.214 | 86.268 | 15 | 332 | 23 | 277 |
| B. lactis | 273 | 13.355 | 30.328 | 401 | 27.403 | 53.806 | 49 | 111 | 61 | 134 |
| L. paracasei | 361 | 5.881 | 45.341 | 763 | 20.919 | 78.489 | 16 | 126 | 27 | 102 |
| GF | 281 | 8.302 | 58.284 | 256 | 33.744 | 40.64 | 29 | 207 | 131 | 158 |

### Example 2

### Immunosuppressive Effects of Lactobacillus paracasei NCC 2461: Incidence on Oral Tolerance Induction to Bêta-Lactoglobulin

Molecular Size of Native BLG and its TC Fractions Before and After Degradation with L. paracasei Extract.

To assess whether L. paracasei extract hydrolyzes BLG and its TC fractions, molecular size of peptides before and after degradation with L. paracasei extract was compared. Native BLG was not at all degraded by L. paracasei extract while both acidic and basic TC fractions were. Acidic peptides with high molecular weights (1000-5000 Da) were hugely degraded, releasing small peptides with molecular weighs lower than 1000 Da. Small peptides represent more than 75% of the degraded-fraction compared to 36% before, 42% of the released peptides being lower than 500 Da. The basic fraction was degraded to a lesser extent. Peptides ranging from 500 to 1000 Da decreased in the fraction to give peptides smaller than 500 Da, the latest representing 43% of the fraction after degradation compared to 37% before. Surprisingly, peptides with molecular weights higher than 2000 Da increased in the degraded-fraction.

Effect of BLG and its TC Fractions Degraded or not by L. paracasei Extract on Lymphocyte Proliferation.

Native BLG and basic TC fraction have no effect on lymphocyte proliferation even after hydrolysis with L. paracasei extract regardless of concentration. In contrast, acidic TC fraction stimulated lymphocyte proliferation when used at concentrations higher than 1,000 μg/ml as shown by stimulation indices higher than 1.8 and reaching 2.5 at 2,000 μg/ml. After degradation with L. paracasei extract, this stimulating effect was significantly repressed (p<0.05), the strongest suppression being observed at 2,000 μg/ml. In addition, the immunosuppressive effect of L. paracasei-degraded acidic fraction was found more apparent with splenocytes of mice never previously exposed to BLG, showing reduction of stimulation index of 0.7 compared to 0.4 for BLG-primed mice.

Effect of BLG and its TC Fractions Degraded or not by L. paracasei Extract on Cytokine Production.

Low levels of IFN-γ and IL-10 were produced by splenocytes in response to native BLG and basic TC fraction degraded or not with L. paracasei extract. The acidic TC fraction was found to induce high level of IFN-γ and low level of IL-10 while its degradation with L. paracasei extract induces the opposite response. IL 4 was found to be produced at low levels regardless of fraction but degradation of BLG and acidic fraction reduced its secretion at levels close to the detection limit of the test.

Effect of L. paracasei Extract on Lymphocyte Proliferation and Cytokine Production.

To better understand the mechanism by which L. paracasei can induce oral tolerance to BLG in mice, the effect of the cytoplasmic extract alone on lymphocyte proliferation and cytokine production has been investigated. L. paracasei extract suppressed the mitogen-induced lymphocyte proliferation in dose dependant manner with maximal suppression at low dilution (1/10) and no effect at high dilution (1/1000). The cell proliferation observed in absence of PHA was not affected by L. paracasei extract even at high dose (dilution 1/10) suggesting that the concentrations used were not toxic for cells. Moreover, a large amount of IL-10 was produced when cell proliferation was strongly repressed while production of IFN-γ was not affected and remained very low.

### Example 3

### Maintenance of Oral Tolerance

The experiment is the continuation of example 1.

The maintenance of specific antibody response suppression in tolerized mice was monitored for a 7-week period after whey proteins feeding (FIG. 1). BLG-specific IgE, IgG1 and IgG2a responses were maintained suppressed in conventional mice (p<0.01). The suppression of anti-BLG IgG2a titer in germ free and L. paracasei-associated mice was not maintained, whereas both specific IgE and IgG1 levels were. A continued suppression of BLG-specific IgE responses and a significant decrease anti-BLG IgG1 titer were observed in mice colonised with B. lactis. As result, Th1- and Th2-type humoral responses were maintained suppressed in conventional mice, while only Th1-type antibody titers were maintained abrogated in germfree, B. lactis and L. paracasei-associated mice.

IFN-γ productions by spleen cells in mice sacrificed 50 days after oral tolerance induction were similar to those obtained after 28 days, except for germfree and B. lactis-associated mice for which the decrease became not significant and significant, respectively (FIG. 2B). The Th1-cell activity also remained suppressed in all groups of mice (FIG. 2B). In contrast, the high production of IL-10 was maintained only in conventional mice given whey proteins (FIG. 2B). Nevertheless, spleen cells from germfree and B. lactis-associated mice have been found to be sensitive to long-term tolerance persistence clearly shown by high stimulation indexes reaching 131, 61 and 38, respectively, in presence of BLG (Table 1). Lower stimulation indexes were obtained in conventional mice and mice colonized with L. paracasei in presence of BLG, while cells from all the groups of mice responded well to PHA (Table 1).

### Example 4

### Anti-Allergy Proeperties of Bifidobacterium lactis Bb12 Against Bêta-Lactoglobulin

Characterization of BLG TC Fractions—Effect of B. lactis-Driven Hydrolysis on the Molecular Size of BLG and its TC Peptides.

Both acidic and basic fractions were degraded by B. lactis extract while native BLG did not. Before B. lactis degradation, the acidic fraction was composed of 36% peptides with molecular weights ranging from 2000 to 5000 Da After B. lactis degradation, the percentage of these peptides decreased to 21% while low molecular weight peptides (<500 Da) increased from 19% to 46%. Small peptides were also released after basic fraction hydrolysis. Basic peptides with molecular weights from 500 to 2000 Da were breakdown to give small peptides with delayed retention time.

IgE Binding Capacity of BLG and its TC Fractions

Binding capacity of BLG and its basic TC fraction is not significantly reduced by degradation with B. lactis extract. In contrast, serum IgE binding by acidic TC peptides was slightly reduced by B. lactis-driven hydrolysis.

Effect of BLG and its TC fractions Degraded by B. lactis Extract on Lymphocyte Proliferation

Before B. lactis degradation, lymphocyte proliferation was significantly stimulated in the presence of acidic fraction at concentrations of 1,000 μg ml ⁻¹ or more but not in the presence of BLG and basic fraction regardless of concentration. In contrast, after degradation with B. lactis extract, BLG and its TC fractions all stimulated lymphocyte proliferation in a dose dependent manner. BLG and basic fraction induced significant cell proliferation at concentrations equal to or higher than 1,000 μg ml ⁻¹ while acidic fraction did so at a concentration of 2,000 μg ml ⁻1. Moreover, stimulation indices obtained at 2,000 μg ml ⁻¹ were similar for BLG, acidic and basic fractions. Although acidic and basic TC peptides had distinct stimulating effects, degradation with B. lactis extract produced converging effects at high dose.

Since BLG hydrolyzed by B. lactis extract, unlike acidic and basic fractions, was not subsequently filtered to remove enzymes, the effect of the extract alone on lymphocyte proliferation was assessed as a control. Diluted to 1/100 (as for BLG hydrolysis, wells containing 2000 μg ml ⁻¹ of BLG also containing 40 μg ml ⁻¹ of cell extract protein), the extract strongly stimulated lymphocyte proliferation suggesting that the stimulating effect of BLG may have been due mainly to cell-free extract present at 40 μg ml ⁻¹. In contrast, ultrafiltration of the B. lactis extract itself produced permeate essentially free of proteins as verified by C18 chromatography suggesting that only traces of the extract was remaining in both acidic and basic fractions after filtration. At low concentration (1/1000 or 4 μg ml ⁻1, of cell extract protein), the extract did not likely stimulate lymphocyte proliferation indicating no contribution to the observed effect of acidic and basic fractions.

Proliferation of Lymphocyte from Naïve and Tolerant Mice

This paragraph deals with effect of B. lactis extract on the factors used to stimulate in vitro proliferation of lymphocytes from young naïve mice and mice daily receiving a BLG-enriched diet. Before degradation by B. lactis extract, the acidic fraction stimulated lymphocytes from naïve mice more than those from their tolerant counterparts while BLG and basic peptides did not. After degradation with B. lactis extract, BLG and its TC fractions all stimulated significantly stronger proliferation of lymphocytes from naïve mice compared to tolerant mice as indicated by indices reaching 4 compared to 3. However, the B. lactis extract alone (dilution 1/100) was shown to stimulate lymphocyte proliferation in naïve mice as previously mentioned for tolerant mice, suggesting no real stimulating effect of native BLG degraded by B. lactis extract on proliferation.

Cytokine Production

This paragraph deals with the production of cytokines by splenocytes from naïve and tolerant mice in the presence of BLG and its TC fractions, hydrolyzed or not by B. lactis extract. In naïve and tolerant mouse splenocyte cultures, IFN-γ was produced at low levels in the presence of untreated BLG, acidic and basic TC fractions, with the highest production of 400 pg ml ⁻¹ obtained in response to acidic fraction. When hydrolyzed by B. lactis extract, BLG and fractions strongly induced IFN-γ secretion by both naïve and tolerant mouse splenocytes with higher production for naïve mice. The same profile of response was observed for IL-10 secretion but the levels of production were ten times lower than for IFN-γ. No IL-4 or IL-5 was detected in cell culture supernatants regardless of BLG sample or mice, suggesting no strong Th2-type cytokine induction. As observed for lymphocyte proliferation, the production of IFN-γ in response to BLG degraded by B. lactis extract is due mainly to extract remaining in the sample.

### Example 5

### Infant Formulae Promoting and Inducing Oral Tolerance

We prepare an infant formula by mixing together the following ingredients in the indicated proportions. The final product comprises 519 kcal for 100 g, and is in powder form.

| | |
|---|---|
| Fat | 27.7 g |
| Fat from milk | 0.7 g |
| Mixture of fats (150) | 26.8 g |
| Lecithin | 0.2 g |
| Linoleic acid | 4.1 g |
| α-linolenic acid | 525 mg |
| Proteins | 9.5 g |
| Free carbohydrates | 57.9 g |
| Lactose | 57.9 g |
| Minerals (ashes) | 1.9 g |
| Sodium | 120 mg |
| Potassium | 460 mg |
| Chlorure | 330 mg |
| Calcium | 320 mg |
| Phosphorus | 160 mg |
| Magnesium | 36 mg |
| Manganese | 40 μg |
| Selenium | 10.4 μg |
| Total solids | 97.0 g |
| Humidity | 3.0 g |

We then add to the above-prepared formulae 106 per 100 g of Lactobacillus paracasei, and 10² of Bifidobacterium lactis.

### Example 6

### Cat Food Promoting and Inducing Oral Tolerance

A feed mixture is made up of about 58% by weight of corn, about 6% by weight of corn gluten, about 23% by weight of chicken meal, salts, vitamins and minerals making up the remainder.

The feed mixture is fed into a preconditioner and moistened. The moistened feed is then fed into an extruder-cooker and gelatinised. The gelatinised matrix leaving the extruder is forced through a die and extruded. The extrudate is cut into pieces suitable for feeding to cats, dried at about 110° C. for about 20 minutes, and cooled to form pellets. At this point, a lyophilised powder of one or more strains of the following species is provided for application to the pellets: Lactobacillus paracasei and Bifidobacterium lactis. Sufficient powder is thus provided so that the corresponding dietary intake amount for the cat is from about 10⁷-10⁹ cfu/day. Some of the powder is mixed into a first mass of pellets and bagged. A second quantity of the powder is measured out and mixed with a lipid carrier which is then sprayed on to a second mass of pellets. The pellets are bagged after the coating has dried sufficiently at 50-60° C. for some minutes.

1. A method of manufacturing a food product for the maintenance of oral tolerance to antigens comprising the step of using Bifidobacteria to produce the food product.

2. The method according to claim 1 wherein the Bifidobacterium is a Bifidobacterium lactis.

3. The method according to claim 1 wherein the Bifidobacterium is Bifidobacterium lactis ATCC 27536.

4. The method according to claim 1 wherein the added bacteria are inactivated or dead.

5. The method according to claim 1 wherein the food product is an infant formula.

6. The method according to claim 1 wherein the bacteria are present in the food product in an amount of 10⁵, to 10⁸, cfu/mL.

7. The method according to claim 1 wherein the food product is a pet food.

8. The method according to claim 7 wherein the bacteria are present in an amount of 10⁴ and 10⁸ cfu/g.

9. A method for the manufacture of a food product for the promotion and maintenance of oral tolerance to antigens comprising the steps of using a Lactobacillus and a Bifidobacterium to manufacture the food product.

10. The method according to claim 9 wherein the Lactobacillus is a Lactobacillus paracasei and the Bifidobacterium is a Bifidobacterium lactis.

11. The method according to claim 10 wherein the Lactobacillus paracasei is Lactobacillus paracasei CNCM I-2116 and the Bifidobacterium lactis is Bifidobacterium lactis ATCC 27536.

12. The method according to claim 9 wherein the food product is an infant formula.

13. The method according to claim 12 wherein the Lactobacilli:Bifidobacteria ratio is comprises between 10:1 and 1:1.

14. The method according to claim 12 wherein the Lactobacilli:Bifidobacteria ratio comprises between 1:10 and 1:1.

15. The method according to claim 9 wherein the food product is a pet food.

16. The method according to claim 9 wherein the amount of microorganisms in the food product is comprised between 10⁴ to 10⁸ cfu/g of food product or cfu/mL.

17. A method for decreasing the risk of rejecting transplants in an individual comprising the steps of administering to an individual having a transplant a food product comprising a therapeutically-effective amount of a probiotic.

18. The method according to claim 17 wherein the food product comprises a Lactobacillus to induce oral tolerance, and a Bifidobacterium to maintain an oral tolerance previously induced.

19. The method according to claim 18 wherein Lactobacillus is Lactobacillus paracasei, preferably Lactobacillus paracasei CNCM I-2116.

20. The method according to claim 19 wherein the Bifidobacterium is a Bifidobacterium lactis, preferably Bb 12 strain ATCC 27536.

21. The method according to claim 1 wherein the bacteria are present in the food product in an amount of 10⁵ to 10⁷ cfu/mL.

22. The method according to claim 1 wherein the bacteria are present in the food product in an amount of 10⁶ cfu/mL.

23. The method according to claim 7 wherein the bacteria are present in the food product in an amount of 10⁵ and 10⁸ cfu/g.

24. The method according to claim 23 wherein the food product is a liquid product.

25. The method according to claim 9 wherein the amount of microorganisms in the food product comprises between 10⁴ to 10⁹ cfu/g.

26. A method for the promotion or maintenance of oral tolerance to an antigen in an infant comprising the step of administering to the infant a product comprising a Lactobacillus and a Bifidobacterium.

27. The method according to claim 26 wherein the product is administered to the infant after contact with an antigen.

28. The method according to claim 26 wherein the product is administered to the infant prior to contact with an antigen.

29. The method according to claim 26 wherein the Lactobacillus is a Lactobacillus paracasei and the Bifidobacterium is a Bifidobacterium lactis.

30. The method according to claim 26 wherein the Lactobacillus paracasei is Lactobacillus paracasei CNCM I-2116 and the Bifidobacterium lactis is Bifidobacterium lactis ATCC 27536.
